(19) United States
(12) Patent Application Publication
(10) Pub. No.: US 2013/0092171 A1
(43) Pub. Date: Apr. 18, 2013

(54) MULTI-LUMEN TRACHEAL TUBE WITH PRESSURE DISTRIBUTION

(71) Applicants: Donn Sederstrom, West Linn, OR (US); Tyler Grubb, Portland, OR (US); Alexa Jansey, Culver, IN (US); Christopher Brune, Evergreen, CO (US); Corinne Lengsfeld, Denver, CO (US); Brian Rosenkrans, Lyons, CO (US)

(72) Inventors: Donn Sederstrom, West Linn, OR (US); Tyler Grubb, Portland, OR (US); Alexa Jansey, Culver, IN (US); Christopher Brune, Evergreen, CO (US); Corinne Lengsfeld, Denver, CO (US); Brian Rosenkrans, Lyons, CO (US)

(73) Assignee: Nellcor Puritan Bennett LLC, Boulder, CO (US)

(21) Appl. No.: 13/274,996

(22) Filed: Oct. 17, 2011

(51) Int. Cl. A61M 16/04; B23P 17/04

(52) U.S. Cl. 128/207.15; 29/428

(57) ABSTRACT

The present disclosure describes systems and methods that utilize a tracheal tube with pressure distribution features. For certain patients, the vocal cords may form a seal with an inserted tracheal tube. A pressure distribution lumen may allow fluid communication between tracheal space above and below the vocal cords. This in turn may reduce the formation of a vacuum seal forming around a suction lumen evacuation port. Accordingly, the disclosed embodiments provide improved suctioning by distributing the pressure around the evacuation port and the space above the vocal cords.

### BACKGROUND

The present disclosure relates generally to medical devices and, more particularly, to tracheal tubes that include pressure distribution features.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the course of treating a patient, a tracheal tube (e.g., endotracheal, nasotracheal, or transtracheal device) may be used to control the flow of gases into the trachea of a patient. Often, a seal between the outside of the tube and the interior wall of the tracheal lumen is required, allowing for generation of positive intrathoracic pressure distal to the seal. Such seals may be formed by inflation of a balloon cuff inside the trachea that contacts the tracheal walls.

The tracheal seal may also prevent or reduce ingress of solid or liquid matter into the lungs from proximal to the seal. In particular, normal swallowing and draining activities of the upper respiratory tract may be disrupted by intubation. Accordingly, secretions (e.g., mucus and saliva) formed in the mouth may gather and pool above a shelf formed by the inflated tracheal cuff. To reduce any migration of this material past the seal of the cuff and into the lungs, clinicians may manage the accumulation of secretions around the seal of the cuff via external suctioning. For example, some tracheal tubes include a dedicated lumen formed in the wall of the tracheal tube that includes a port or opening configured to access any pooled secretions. When negative pressure is applied to the lumen, for example via a syringe, the secretions enter the lumen through the port and are removed from the patient.

### BRIEF DESCRIPTION OF THE DRAWINGS

Advantages of the disclosed techniques may become apparent upon reading the following detailed description and upon reference to the drawings in which:

FIG. 1 is a perspective view of a tracheal tube with a pressure distribution lumen inserted into a patient in accordance with embodiments of the present disclosure;

FIG. 2 is a perspective view of the tracheal tube of FIG. 1;

FIG. 3 is a partial perspective view of an inserted tracheal tube with a pressure distribution lumen in accordance with embodiments of the present disclosure;

FIG. 4 is a partial perspective view of a tracheal tube with a pressure distribution lumen with embodiments of the present disclosure;

FIG. 5 is cross-sectional view of a tracheal tube with pressure distribution lumen in accordance with embodiments of the present disclosure;

FIG. 6 is a perspective view of an alternative tracheal tube a pressure distribution lumen with multiple ports; and

FIG. 7 is a perspective view of an alternative tracheal tube with multiple pressure distribution lumens.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Tracheal tubes form a seal against the tracheal walls at a location below the vocal chords. Typically, the tracheal tube is inserted until its distal end is beyond the vocal cords but some distance above the patient's carina, which allows the cuff to form a seal at an appropriate location in the lower airway space. For some patients, an addition seal is formed above (i.e., proximal to) the cuff by interaction of the exterior of the tracheal tube and the tracheal tissue, particularly around the vocal cords, which protrude into the airway. Because the airway narrows around the vocal cords, some patients may have an airway diameter in the vocal cord region that is close to the external diameter of the tracheal tube, which may cause at least a partial seal to form. Further, certain clinical conditions may cause swelling of the vocal cords, which may also contribute to this effect. When a seal forms around the tracheal tube and the vocal cords (or any other protruding airway region proximal of the cuff), air is trapped between the cuff seal and the vocal cord seal. This sealed region of trapped air may interfere with the ability of clinicians to suction secretions that accumulate above the cuff. That is, if the suction lumen opens to an evacuation port that is positioned within this sealed region, a suction force may be unable to pull negative pressure through the lumen to draw secretions into the evacuation port once the trapped air is removed. Even in embodiments in which a vacuum is not created once the trapped air is removed (e.g., when only a partial seal is formed around the vocal cords), the difference in pressure between the two seals and the space in the upper airway may reduce the effectiveness of suctioning.

As described in detail below, embodiments of tracheal tubes having a pressure distribution lumen are provided herein. In particular, the disclosed tracheal tubes include one or more dedicated suction lumens for removal of secretions as well as one or more dedicated pressure distribution lumens that provide fluid communication between an area between the vocal cords and the cuff and an area above the vocal cords, thus equalizing the pressures between these areas. The pressure distribution lumen may reduce the likelihood of trapped air between a cuff seal and a seal formed around the vocal cords, which in turn may improve the suctioning function of the suction lumen. Further, the reduction in formation of a double seal may also reduce the likelihood of accidental obstruction of the evacuation port by tissue suction that may occur if the clinician attempts to establish negative pressure through the suction lumen. That is, when the clinician removes the relatively small volume of trapped air, the negative pressure may pull in tracheal wall tissue towards the evacuation port. In addition, the tracheal tubes with pressure distribution lumens may allow clinicians to accurately determine if an evacuation port is blocked by a physical obstruction, as opposed to being blocked because the formation of a sealed region has prevented the drawing of mucus or secretions through the suction lumen. In such cases, if a clinician may more confidently determine that the lumen is physically obstructed, then appropriate steps may be taken that will clear the obstruction (e.g., flushing the lumen with fluid or positive pressure).

The tracheal tubes as provided herein are disposable rather than reusable, capable of providing differential mechanical ventilation to either or both lungs, and capable of supporting all other functions of standard endotracheal tubes (e.g. sealing, positive pressure generation, suctioning, irrigation, drug instillation, etc). The tracheal tubes can be used in conjunction with all acceptable auxiliary airway devices such as (e.g. heat and humidity conservers, mechanical ventilators, humidifiers, closed suction systems, scavengers, capnometers, oxygen analyzers, mass spectrometers, PEEP/CPAP devices, etc). Furthermore, although the embodiments of the present disclosure illustrated and described herein are discussed in the context of tracheal tubes such as endotracheal tubes, it should be noted that presently contemplated embodiments may include a pressure distribution lumen used in conjunction with other types of airway devices. For example, the disclosed embodiments may be used in conjunction with a single-lumen tube, tracheostomy tube, a double-lumen tube (e.g., a Broncho-Cath™ tube), a specialty tube, or any other airway device with a main ventilation lumen. Indeed, any device with a ventilation lumen designed for use in an airway of a patient may include a pressure distribution lumen. As used herein, the term “tracheal tube” may include an endotracheal tube, a tracheostomy tube, a double-lumen tube, a bronchoblocking tube, a specialty tube, or any other airway device. In addition, the pressure distribution lumen may be incorporated into catheters or other inserted or implantable medical devices. Further, although the disclosed embodiments related to pressure equalization below and above the vocal cords, the pressure distribution lumen may be configured to equalize pressure between other types of anatomical structures that may form undesired seals in an area proximal or distal of an inflatable cuff.

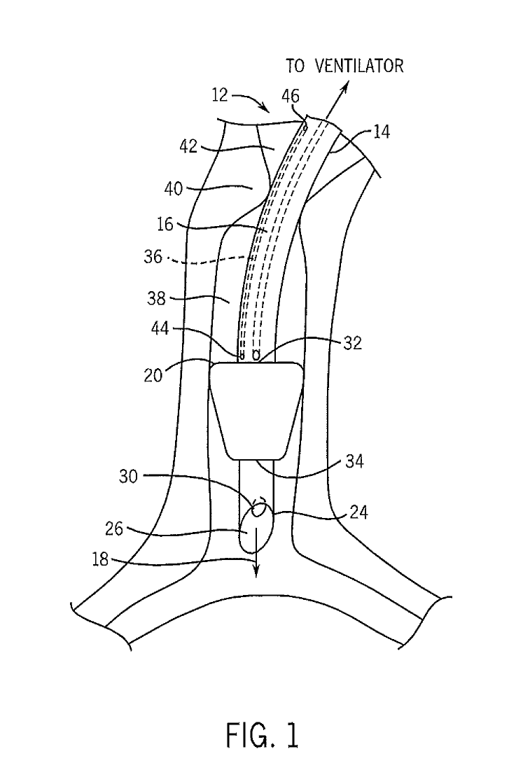

Turning now to the drawings, FIG. 1 is a perspective view of an exemplary tracheal tube 12 with pressure distribution features and configured to be placed in a patient's airway in accordance with aspects of the present disclosure. The tracheal tube 12 includes a central tubular body 14 that defines a ventilation lumen 16 that facilitates the transfer of gases to and from the lungs, e.g., as airflow into the lungs shown by arrow 18. The tracheal tube 12 includes an inflatable cuff 20 disposed towards a distal end 24. The distal end 24 terminates in an opening 26. A proximal end of the tracheal tube 12 may connect to upstream airway devices (e.g., a ventilator). A Murphy eye 30 may be located on the tubular body 14 opposite the opening 26 to prevent airway occlusion when the tracheal tube 12 is improperly placed within the patient's trachea.

The cuff 20 is configured to seal the tracheal space once inflated against the tracheal walls. The cuff 20 is typically affixed to the tubular body 14 via a proximal shoulder 32 and a distal shoulder 34. As noted, the present disclosure relates to tracheal tubes with pressure distribution features. For example, the tracheal tube 12 may include a pressure distribution lumen 36 that facilitates fluid communication between a space 38 below the vocal cords 40 and a space 42 above the vocal cords 40. In the depicted embodiment, the pressure distribution lumen includes a first opening 44 and a second opening 46 that provide fluid communication between the space 38 and the space 42. In particular, depending on individual patient anatomy, the tubular body 14 may form a seal against the vocal cords 40, which results in a fluid separation between the space 38 and the space 42. In the disclosed embodiments, the pressure distribution lumen 36 allows air to flow between the space 38 and the space 42. In certain embodiments, patients with enlarged or irritated vocal cords may be more likely to form such vocal cord seals. In other embodiments, the seals may be likely to form based on the patient anatomy, the outer diameter of the selected tracheal tube, and the size of the vocal cords.

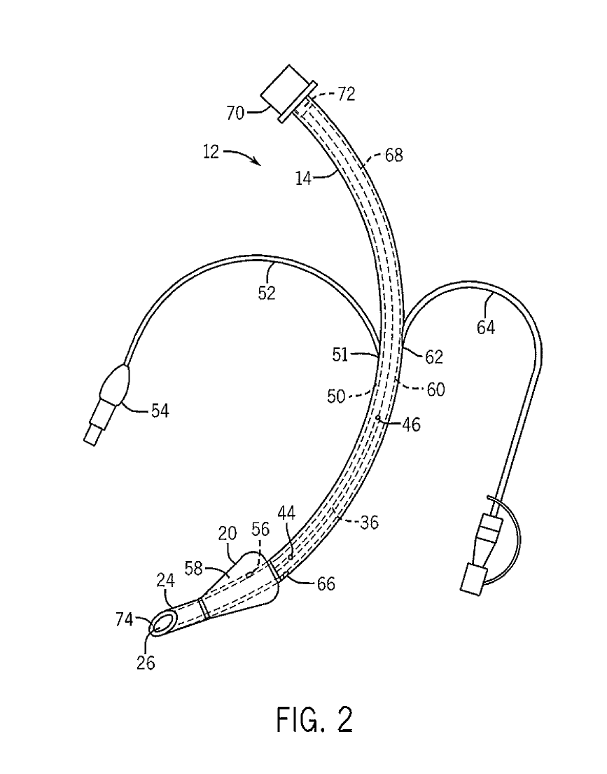

As shown in greater detail in perspective view in FIG. 2, the tracheal tube 12 may include separate dedicated lumens for cuff inflation, suction, and pressure distribution. For example, the cuff 20 may be inflated via inflation lumen 50 that emerges from the tubular body 14 at a junction 51 and terminates at its proximal end in an inflation tube 52 connected to an inflation pilot balloon and valve assembly 54. The inflation lumen 50 terminates in notch 56, which is in fluid communication with the interior space 58 of the cuff 20. Additionally, it should be noted that the cuff 20 may be any suitable cuff, such as a tapered cuff, a non-tapered cuff, and so forth. The tracheal tube 12 may also include a suction lumen 60 that extends along the tracheal tube 12 and emerges from a junction 62 on the tracheal tube 12 proximal of the vocal cords 40 (see FIG. 1) to a suction line 64. The suction lumen 60 is in fluid communication with an evacuation port 66 for suctioning secretions into the suction lumen 60, and out of the tube via the suction line 64.

The tracheal tube 12 and the cuff 20, as well as any associated lumens, are formed from materials having suitable mechanical properties (such as puncture resistance, pin hole resistance, tensile strength), chemical properties (such as biocompatibility). In one embodiment, the walls of the cuff 20 are made of a polyurethane having suitable mechanical and chemical properties. An example of a suitable polyurethane is Dow Pellethane® 2363-80A. In another embodiment, the walls of the cuff 20 are made of a suitable polyvinyl chloride (PVC). In certain embodiments, the cuff 20 may be generally sized and shaped as a high volume, low pressure cuff that may be designed to be inflated to pressures between about 15 cm H₂O and 30 cm H₂O. However, it should be understood that the intracuff pressure may be dynamic. Accordingly, the initial inflation pressure of the cuff 20 may change over time or may change with changes in the seal quality or the position of the cuff 20 within the trachea. The tracheal tube 12 may be coupled to a respiratory circuit (not shown) that allows one-way flow of expired gases away from the patient and one-way flow of inspired gases towards the patient. The respiratory circuit, including the tracheal tube 12, may include standard medical tubing made from suitable materials such as polyurethane, polyvinyl chloride (PVC), polyethylene teraphthalate (PETP), low-density polyethylene (LDPE), polypropylene, silicone, neoprene, polytetrafluoroethylene (PTFE), or polyisoprene. In addition, the tracheal tube may feature a Magill curve. In one embodiment, the suction lumen 60 and evacuation port 66 may be positioned on an outside surface 68 of the curve, such that the evacuation port 66 generally faces a dorsal side when inserted into the patient. The tracheal tube 12 may also include a connector 70 at its proximal end 72 for connection to upstream devices via appropriate tubing.

The lumens (e.g., ventilation lumen 16, pressure distribution lumen 36, inflation lumen 50, and/or suction lumen 60) may be formed in the tubular body 14 via an extrusion process. In such an implementation, the lumens run alongside the airflow path of the ventilation lumen 16 from the proximal end 72 to the distal end 24. In particular embodiments, the pressure distribution lumen 36, inflation lumen 50, and/or suction lumen 60 are not in fluid communication with the ventilation lumen 16. In one embodiment, one or more lumens of the tube 12 are sealed at the distal end during formation of a shaped or beveled tip 74. For example, a heat shaping process may close any lumens. In addition, the connector 70 may compress the lumens at the proximal end 72, although such compression may or may not result in a seal for the lumens at the proximal end 70.

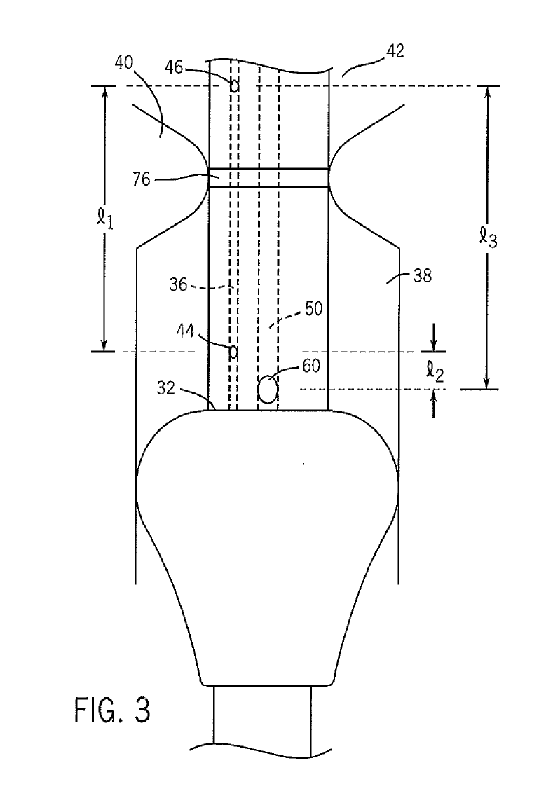

The pressure distribution lumen 36 provides fluid communication between the space 38 and the space 42 to allow more efficient suctioning of secretions through the suction lumen 60 via the evacuation port 66. To that end, the pressure distribution lumen 36 may include at least one opening 44 positioned in the space 38 and at least one opening 46 positioned in the space 42 when the tracheal tube 12 is properly inserted in a patient, as shown in FIG. 3. The first opening 44 and the second opening 46 may be characterized by their separation length, indicated as l₁, along a direction or axis of fluid flow. The separation length l₁ may be selected based on an estimate of the patient's anatomy (i.e., the location of the vocal cords 40) and the tube size. In one embodiment, the first opening 44 and the second opening 46 are spaced apart a length l₁ of at least about 2 cm. In another embodiment, the first opening 44 and the second opening 46 are spaced apart a length l₁ of about 2 cm to about 10 cm. In further embodiments, the length may be larger if, for example, the second opening 46 is positioned outside of the body or on a connector or lumen coupled to the pressure distribution lumen 36 that is capable of providing fluid communication between the space 38 and a space above the vocal cords, e.g., ambient air outside the patient. However, in specific embodiments, it may be advantageous to provide the second opening 46 on the tubular body 14 in the inserted portion of the tube 12 so that the fluid communication is between two spaces that are within the patient's own body. Further, a shorter length l₁ may be advantageous for providing more efficient pressure distribution. Accordingly, in particular embodiments, the length l₁ may be about 2 cm to about 7 cm.

In another embodiment, the first opening 44 and the second opening 46 may be characterized by their position relative to features of the tracheal tube 12 such as the distal end 24 (see FIG. 2), the proximal shoulder 32 of the cuff 22, or any marking lines or structures on the tube 12. For example, the first opening 44 and the second opening 46 may be characterized by their vertical displacement (l₂ and l₃, respectively), from the evacuation port 66 along a direction of fluid flow. In one embodiment, the first opening 44 may be positioned at the level of the evacuation port 66, in which case there is no vertical displacement l₂ between the evacuation port 66 and the first opening 44. In the depicted embodiment, the first opening 44 is displaced a distance l₂ of about 0.5 cm to about 1.5 cm above (i.e., proximal of) the evacuation port. Such an embodiment may provide the advantage of positioning the first opening 44 above the secretions that build up around the evacuation port 66. It should be understood that the distance l₂ may be measured according to any analogous position of the evacuation port 66 and the first opening 44 (or the second opening 46), such as the position of the top of the evacuation port 66 relative to the top of the first opening 44. In addition, certain tracheal tubes 12 may include vocal cord indicators, such as one or more rings 76, that are positioned to be at an estimated vocal cord location when the tube 12 is inserted. In one embodiment, the first opening 44 and the second opening 46 may be positioned on opposing side of any vocal cord indicator marking. In one embodiment, the first opening 44 and the second opening 46 may be positioned at least 1 cm away from such an indicator.

In another embodiment, the positions of the first opening 44 and the second opening 46 of the pressure distribution lumen 36 may be selected with certain aspects of patient anatomy in mind. For example, the distance from the patient's carina to the vocal cords, for an average adult patient, may be estimated as 10 cm to 15 cm. Accordingly, the tube 12 may be configured such that, when properly positioned in the patient, the proximal shoulder 32 of the cuff 20 is about 2 cm below the vocal cords 40. In such an embodiment, the first opening 44 may be positioned along the tubular body 14 within the estimated 2 cm between the vocal cords 40 and the proximal shoulder 32 of the cuff, and the second opening 46 may be positioned in the space estimated to be above the vocal cords 40. Further, the separation l₁ between the first opening 44 and the second opening 46 may be selected based on an estimate of the length of the vocal cords 40 along the trachea. In one embodiment, the vocal cords 40 are estimated to be about 1.5 cm in length. Accordingly, the distance l₁ between the first opening 44 and the second opening 46 may be at least 1.5 cm such that the protrusion of the vocal cords 40 into the tracheal space does not obstruct either the first opening 44 or the second opening 46 and interfere with pressure distribution. It should be understood that, depending on the size, gender, and/or age of the patient, anatomical distance estimates may differ. Accordingly, the position of the first opening 44 and/or the second opening 46 may be selected based on the tube size and the estimated patient population. For example, a smaller tube may feature a shorter distance l₁ between the first opening 44 and the second opening 46. In addition, it should be understood that different size tubes 12 may feature different positions of any vocal cord indicators, such as the ring 76.

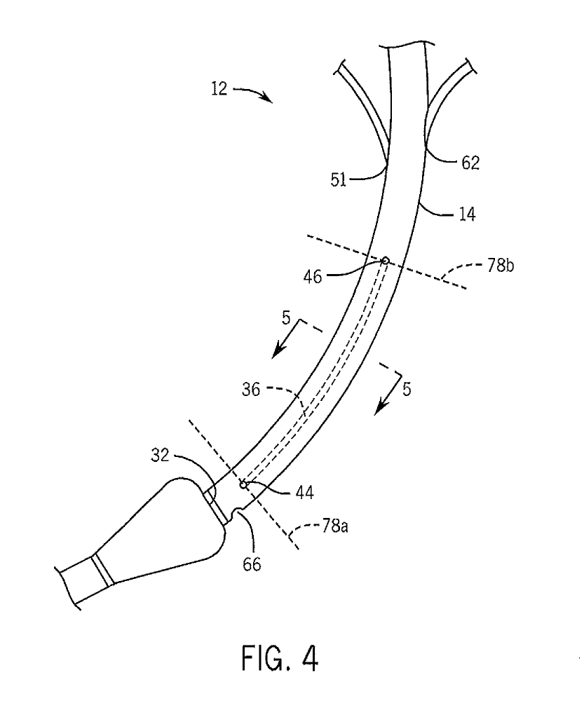

In one embodiment, shown in FIG. 4, both the first opening 44 and the second opening 46 may be positioned with respect to the junction of any external lines with the tubular body 14. For example, the first opening 44 and the second opening 46 may be between the proximal shoulder 32 and the junction 51 (or, alternatively, the junction 62). In addition, it should be understood that, in particular embodiments, the pressure distribution lumen 36 may be configured to include only two openings formed in the tubular body 14 that face the tracheal walls, i.e., that are generally oriented along axis 78 and are substantially orthogonal to the direction of airflow along a given section of the tracheal tube 12. Such an implementation may feature no external structures in direct fluid communication with pressure distribution lumen 36. Because the tracheal tube 12 may already include separate suction and cuff inflation lines, such a configuration may reduce confusion between these structures. Further, such embodiments may reduce manufacturing complexity. In additional embodiments, the pressure distribution lumen 36 may be sealed at the distal end 24 and may be compressed at the proximal end 72 such that fluid communication at the proximal end 72 is blocked. Accordingly, in certain embodiments the pressure distribution lumen 36 may be sealed at both ends and may include only the first opening 44 and the second opening 46 for providing fluid communication.

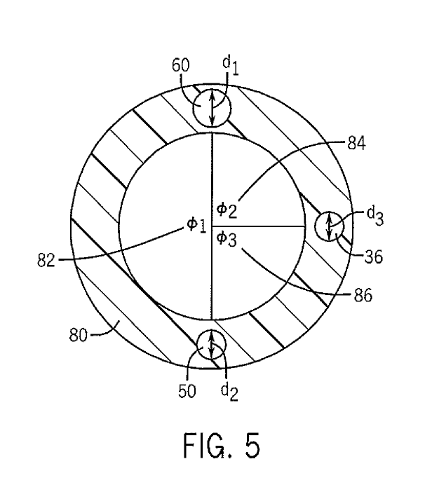

FIG. 5 is a section view of the tracheal tube 12. As noted, the pressure distribution lumen 36, the inflation lumen 50 and the suction lumen 60 are formed within the walls of the tubular body 14. Alternatively, these lumens may be configured as separate structures that are affixed to the exterior of the tubular body 14. As depicted, the pressure distribution lumen 36, the inflation lumen 50 and the suction lumen 60 are circumferentially distributed about the tube wall 80. The position of the pressure distribution lumen 36 may be characterized relative to the suction lumen 60 and/or the cuff inflation lumen 50. For example, in one embodiment, the suction lumen 60 is positioned along the tube 12 such that the evacuation port 66 (see FIG. 4) opens to a dorsal side of the patient when inserted. The pressure distribution lumen 36 may be positioned to open to a ventral side. The pressure distribution lumen 36 may be positioned at any suitable position along the circumference of the tube wall 80. In the depicted embodiment, the cuff inflation lumen 50 is positioned with an angle 82 about 180° away from the suction lumen 60 and the pressure distribution lumen 36 is positioned with an angle 84 about 90° away from the suction lumen 60 and an angle 86 about 90° away from the cuff inflation lumen 50. However, in an alternative embodiment, the pressure distribution lumen may be positioned about 180° away from the suction lumen 60. The separation between the pressure distribution lumen 36, the inflation lumen 50 and the suction lumen 60 may be limited by the inner diameters of these lumens. A pressure distribution lumen 36 may be positioned adjacent to the suction lumen 60, but with sufficient tube wall 80 between the two lumens to preserve the structural integrity of the tubular body 14. In one embodiment, the pressure distribution lumen 36, the inflation lumen 50 and the suction lumen 60 are at least 30° apart from one another along the circumference of the tube wall 80. In another embodiment, the pressure distribution lumen and the suction lumen 60 are between about 30° and about 90° from one another.

The diameter d₁ of the suction lumen 60 may be selected based on appropriate diameters for removing mucus and other secretions from the trachea. For example, the suction lumen 60 may have a diameter d₁ of at least 2.5 mm. Similarly, the cuff inflation lumen 50 may have a diameter d₂ suitable for delivering air to inflate the tracheal cuff. In contrast, the, pressure distribution lumen 36 provides fluid communication between two spaces. Accordingly, while the pressure distribution lumen 36 may have a diameter d₃ that is similar in size to that of the suction lumen 60, the pressure distribution lumen 36 may also have a smaller diameter that reflects its function. For example, the pressure distribution lumen 36 may have a diameter of less than 2 mm or less than lnun. In particular embodiments, a capillary-type pressure distribution lumen 36 may be formed via a wire inserted into tubular body 14 to achieve such diameters.

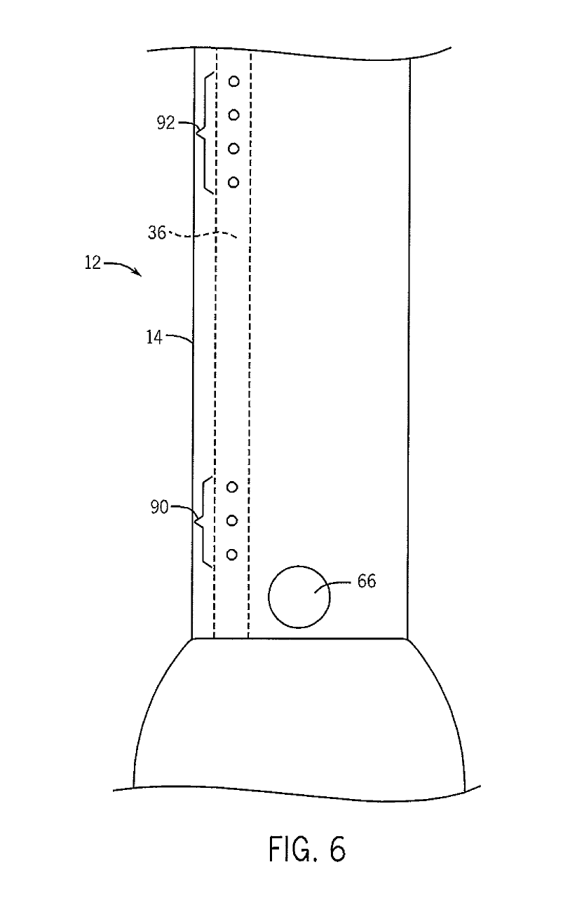

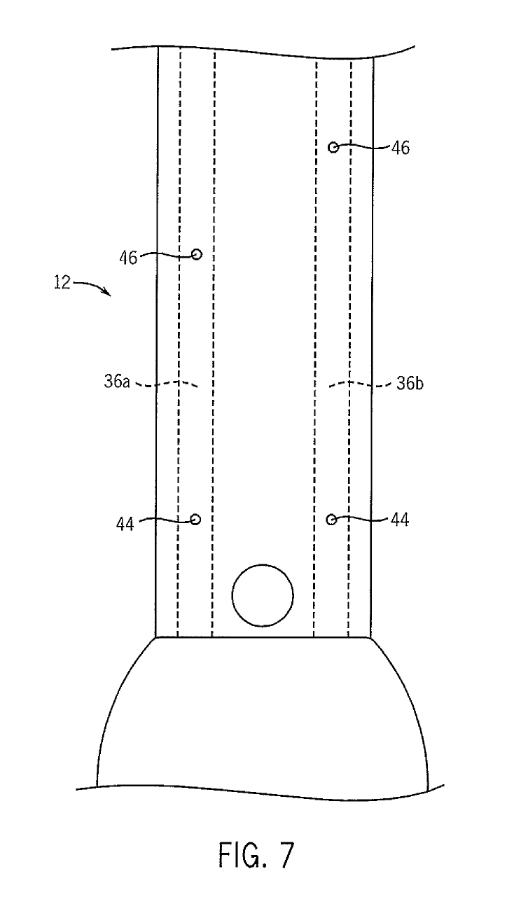

While the pressure distribution lumen 36 may feature only a first opening 44 and only a second opening 46, alternative embodiments may include multiple openings. As shown in FIG. 6, a pressure distribution lumen 36 may include a plurality of openings 90 configured to be positioned below the vocal cords and a plurality of openings 92 configured to be positioned above the vocal cords. In addition, the pressure distribution lumen 36 may feature openings that have different shapes and/or sizes. For example, while a distal opening (e.g., first opening 44) may be positioned relatively close to the evacuation port 66, which may limit its size, a more proximal opening (e.g., second opening 46) may be located on an area of the tubular body 14 that does not include other openings and, thus, may be larger. In other embodiment, shown in FIG. 7, a tracheal tube 12 may include multiple pressure distribution lumens 36a and 36b. The positions of the first opening 44 and the second opening 46 may be staggered between the pressure distribution lumens 36 to provide redundancy.

In one example of the disclosed embodiments, a pressure distribution apparatus was tested in an artificial trachea system. A tracheal tube including a suction lumen and evacuation port was positioned in the artificial trachea. A cork was used to model the presence of vocal cords and to create an artificial seal. The system was coupled to a Model 494 pressure regulator (L.J. Engineering, Huntington Beach, Calif.). In one experiment, an additional lumen open to atmospheric air and open to an area between the cork and the inflated cuff was placed alongside the tracheal tube. Other runs involved clamping the additional lumen to test for conditions without pressure distribution to break the seal. During the testing, mucus was added to cover an evacuation hole (about 2.4 mL). The system was operated at high suction (150 mmHg/3.3 v input control signal). The steps were performed 2-8 two more times for a total of three trials.

TABLE 1
Results from testing

| | With | |
|---|---|---|
| | additional | Clamped |
| | lumen | lumen |
| Trial 1 - Volume of mucus evacuated (mL) | 1.409 | 0.165 |
| Trial 2 - Volume of mucus evacuated (mL) | 1.275 | 0.039 |
| Trial 3 - Volume of mucus evacuated (mL) | 1.433 | 0.069 |

The results of the testing indicated that, when a seal was created, mucus evacuation was reduced. In addition, by adding an additional lumen for pressure distribution, the seal was broken and mucus secretion was improved.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims. Further, it should be understood that elements of the disclosed embodiments may be combined or exchanged with one another.

What is claimed is:

1. A tracheal tub; comprising:
a conduit defining a passageway for delivering gas to a patient's lungs comprising a proximal end and a distal end;
an inflatable balloon cuff coupled to a wall of the conduit;
a suction lumen formed in the wall of the conduit, wherein the suction lumen terminates in an evacuation port formed in the wall of conduit and proximal of the inflatable balloon cuff; and
a pressure distribution lumen formed in the wall of the conduit, wherein the pressure distribution lumen is coupled to a first opening in the wall of the conduit and a second opening in the wall of the conduit spaced apart from the first opening, wherein the first opening and the second opening are proximal of the inflatable balloon cuff and wherein the second opening is proximal of the evacuation port such that the first opening is located below a patient's vocal cords and the second opening is above the patient's vocal cords when the tracheal tube is inserted in the patient.

2. The tracheal tube of claim 1, wherein the first opening is proximal of the evacuation port.

3. The tracheal tube of claim 1, wherein the pressure distribution lumen is sealed at the distal end of the tracheal tube.

4. The tracheal tube of claim 1, wherein the pressure distribution lumen is sealed at the proximal end of the tracheal tube.

5. The tracheal tube of claim 1, wherein the pressure distribution lumen has a smaller internal diameter than the suction lumen.

6. The tracheal tube of claim 1, wherein the pressure distribution lumen has an internal diameter less than 1 mm.

7. The tracheal tube of claim 1, wherein the first opening is smaller than the second opening.

8. The tracheal tube of claim 1, wherein the pressure distribution lumen is distributed about a circumference of the tracheal tube at least 30 degrees apart from the suction lumen.

9. The tracheal tube of claim 1, wherein the pressure distribution lumen is distributed about a circumference of the tracheal tube at least 30 degrees apart from the suction lumen.

10. The tracheal tube of claim 9, wherein the suction lumen is positioned on the tracheal tube such that the evacuation port opens to a dorsal side of the patient when the tracheal tube is inserted.

11. The tracheal tube of claim 1, wherein the first opening and the second opening of the pressure distribution lumen are spaced apart at least two cm from one another along a direction of airflow.

12. The tracheal tube of claim 1, wherein the tracheal tube comprises an indicator that estimates a vocal cord position, and wherein the first opening is located less than 1 cm distally of the indicator.

13. The tracheal tube of claim 1, wherein the tracheal tube comprises an indicator that estimates a vocal cord position, and wherein the second opening is located between the indicator and a location where the cuff inflation lumen emerges from the tracheal wall.

14. The tracheal tube of claim 1, wherein the pressure distribution lumen comprises no additional openings located on the wall of the tracheal tube and substantially orthogonal to an air flow path of the conduit.

15. A tracheal tube, comprising:
a conduit defining a passageway for delivering gas to a patient's lungs comprising a proximal end and a distal end;
an inflatable balloon cuff coupled to a wall of the conduit;
a suction lumen formed in the wall of the conduit, wherein the suction lumen terminates in an evacuation port formed in the wall of conduit and proximal of the inflatable balloon cuff; and
a pressure distribution lumen formed in the wall of the conduit, wherein the pressure distribution lumen is configured to provide fluid communication between an area below the vocal cords and an area above the vocal cords, and wherein the pressure distribution lumen comprises only two openings and is sealed at the distal end.

16. The tracheal tube of claim 15, wherein the first opening is smaller than the second opening.

17. The tracheal tube of claim 15, wherein the pressure distribution lumen is distributed about a circumference of the tracheal tube at least 30 degrees apart from the suction lumen.

18. The tracheal tube of claim 15, wherein the first opening and the second opening are spaced apart at least two cm from one another.

19. A method of manufacturing a tracheal tube, comprising:
providing a conduit defining a passageway for delivering gas to a patient's lungs comprising a proximal end and a distal end, wherein the conduit comprises a suction lumen formed and a pressure distribution lumen formed in a wall of the conduit;
affixing an inflatable balloon cuff to a wall of the conduit;
forming an evacuation port in the wall of conduit in fluid communication with the suction lumen; and
forming a first opening in the wall of the conduit and a second opening in the wall in fluid communication with the pressure distribution lumen, wherein the first opening is proximal of the inflatable balloon cuff and wherein the second opening is proximal of the evacuation port such that the first opening is located below a patient's vocal cords and the second opening is above the patient's vocal cords when the tracheal tube is inserted in the patient.

20. The method of claim 19, comprising sealing the pressure distribution lumen and the suction lumen at the distal end of the tracheal tube.
